SEQUENCE LISTING (9 sequences)

SEQ ID NO: 1 - DNA, 642 bp, Artificial Sequence
  Synthetic
caggctgttgtgactcaggaatctgcactcaccacatcacctggtggaacagtcatactcacttgtcgctcaagtactggggctgttacaactagtaactatgccaactggatacaagaaaaaccagatcatttattcactggtctaatcggtggtaccagcaaccgagctccaggtgttcctgtcagattctcaggctccctgattggagacaaggctgccctcaccatcacaggggcacagactgaggatgatgcaatgtatttctgtgctctatggttcagaaaccattttgttttcggcggtggaaccaaggtcactgtcctaggtcagcccaagtccactcccactctcaccgtgtttccaccttcctctgaggagctcaaggaaaacaaagccacactggtgtgtctgatttccaacttttccccgagtggtgtgacagtggcctggaaggcaaatggtacacctatcacccagggtgtggacacttcaaatcccaccaaagagggcaacaagttcatggccagcagcttcctacatttgacatcggaccagtggagatctcacaacagttttacctgtcaagttacacatgaaggggacactgtggagaagagtctgtctcctgcagaatgtctc

SEQ ID NO: 2 - protein, 213 aa, Artificial Sequence
  Synthetic
QAVVTQESALTTSPGGTVILTCRSSTGAVTTSNYANWIQEKPDHLFTGLIGGTSNRAPGVPVRFSGSLIGDKAALTITGAQTEDDAMYFCALWFRNHFVFGGGTKVTVLQPKSTPTLTVFPPSSEELKENKATLVCLISNFSPSGVTVAWKANGTPITQGVDTSNPTKEGNKFMASSFLHLTSDQWRSHNSFTCQVTHEGDTVEKSLSPAECL

SEQ ID NO: 3 - DNA, 340 bp, Artificial Sequence
  Synthetic
cagcagaaggtgcagcagagcccagaatccctcattgtcccagagggagccatgacttctctcaactgcactttcagcgacagtgcttctcagtattttgcatggtacagacagcaatctgggaaagcccccaaggcactgatgtccatcttctccaatggtgaaaaagaagaaggcagattcacaattcacctcaataaagccagtctgcatttctcgctacacatcagagactcccagcccagtgactctgctctctacctctgtgcagtgaggggccctaatacaggaaactacaaatacgtctttggagcaggtaccagactgaaggttatagcag

SEQ ID NO: 4 - protein, 113 aa, Artificial Sequence
  Synthetic
QQKVQQSPESLIVPEGAMTSLNCTFSDSASQYFAWYRQQSGKAPKALMSIFSNGEKEEGRFTIHLNKASLHFSLHIRDSQPSDSALYLCAVRGPNTGNYKYVFGAGTRLKVIA

SEQ ID NO: 5 - DNA, 343 bp, Artificial Sequence
  Synthetic
gaggctgcagtcacccaaagcccaagaaacaaggtggcagtaacaggaggaaaggtgacattgagctgtaatcagactaataaccacaacaacatgtactggtatcggcaggacacggggcatgggctgaggctgatccattattcatatggtgctggcagcactgagaaaggagatatccctgatggatacaaggcctccagaccaagccaagagaacttctccctcattctggagttggctaccccctctcagacatcagtgtacttctgtgccagcggtgatgcgggacaggggcactccgactacaccttcggctcagggaccaggcttttggtaataa

SEQ ID NO: 6 - protein, 114 aa, Artificial Sequence
  Synthetic
EAAVTQSPRNKVAVTGGKVTLSCNQTNNHNNMYWYRQDTGHGLRLIHYSYGAGSTEKGDIPDGYKASRPSQENFSLILELATPSQTSVYFCASGDAGQGHSDYTFGSGTRLLVI

SEQ ID NO: 7 - DNA, 596 bp, Artificial Sequence
  Synthetic
aacttgtttattgcagcttataatggttacaaataaagcaatagcatcacaaatttcacaaataaagcatttttttcactgcattctagttgtggtttgtccaaactcatcaatgtatcttatcatgtctggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctcgaagcttcaagaattggtcgacagcaggggactttccgaaggctcgagcctctcggaaagtcccctctgtcgactctagaggatcctctcggaaagtcccctctggatccccgatctggggcagagcatataaggtgaggtaggatcagttgctcctacatttgcttctgacatagttgtgttcagatagatctcgagatccattccggtactgttggtaaa

SEQ ID NO: 8 - DNA, 10 bp, Artificial Sequence
  Synthetic
gggactttcc

SEQ ID NO: 9 - DNA, 10 bp, Artificial Sequence
  Synthetic
ggaaagtccc

(19) United States
(12) Patent Application Publication
(10) Pub. No.: US 2011/0126299 A1
(43) Pub. Date: May 26, 2011

(54) TRIPLE TRANSGENIC MOUSE MODEL OF AUTOIMMUNE DISEASE AND NF-KAPPA B IN VIVO IMAGING

(76) Inventors: Bjarne Bogen, Snaroya (NO); Ludvig Munthe, Fjellhamar (NO); Harald Carlsen, Rasta (NO); Rune Blomhoff, Oslo (NO)

(21) Appl. No.: 12/950,590

(22) Filed: Nov. 19, 2010

Related U.S. Application Data
(60) Provisional application No. 61/262,968, filed on Nov. 20, 2009.

(51) Int. Cl. G01N 33/00; A01K 67/027; C12N 15/87; C40B 30/06; A61K 49/00; A61K 38/02; A61P 29/00; A61P 17/00; A61P 1/00; A61P 13/12; A61P 19/02

(52) U.S. Cl. 800/3; 800/18; 800/22; 506/10; 424/9.2; 514/1.1

(57) ABSTRACT

The present invention provides for monitoring of NF-κB-associated inflammation in mice undergoing Id-driven autoimmune disease. The mice are triple transgenic mice expressing both Id⁺ B cells and Id-specific CD4⁺ T cells as well as a luciferase reporter transgene under NF-κB control. NF-κB activation and nuclear translocation of NF-κB, luciferase activity are repeatedly monitored in intact mice by whole body bioluminescence imaging. Results are corroborated at the cellular level by detection of luciferase protein expression in single cells. The results show that imaging of NF-κB activation permits early detection of subclinical disease as well as tracking of disease development.

The present invention claims the benefit of U.S. Prov. Appl. 61/262,968, filed Nov. 20, 2009, the entire contents of which are incorporated herein by reference.

### FIELD OF THE INVENTION

The present invention relates to the production and use of transgenic mice for research and drug discovery, and in particular to triple transgenic TCR⁺Id⁺Luc⁺ mice.

### BACKGROUND OF THE INVENTION

Despite intense research efforts, the etiology of most autoimmune diseases remains obscure. Recently, CD4⁺ T cells that recognize V region (idiotypic, Id) peptides of antibodies have been described in a number of autoimmune diseases in humans (1-4) such as rheumatoid arthritis (3), systemic lupus erythematosus (SLE)(1,2)and multiple sclerosis (4), as well as in several murine models of autoimmune disease (5-7). However, it has been unclear whether Id-specific CD4⁺ T cells may actually cause autoimmune disease and by which mechanism they could do so.

B cell receptors (BCRs) spontaneously undergo antigen processing, and B cells display Id-peptides on their major histocompatibility complex (MHC) class II molecules; such complexes activate Id-specific T cells (8-12). Conversely, Id⁺ B cells can be helped by Id-specific CD4⁺ T cells and differentiate into antibody (10,13) and autoantibody (13-15) secreting B cells. Such findings have paved the way for the concept of Id-driven T-B collaboration, as first suggested by our group (11,16). Similar models were later proposed by others (6,7). Importantly, Id driven T-B collaboration requires BCR ligation for the germinal center reaction and isotype switching to occur (13). Therefore, since autoantigens are ubiquitously expressed, B cells with autoreactive BCRs are especially prone to partake in Id-driven T-B collaboration, explaining why this type of T-B collaboration is associated with induction of autoantibodies and autoimmune disease (13-15).

T cells are tolerant to abundant germline-encoded V region sequences (17-19), in part due to deletion in the thymus (10,14). Thus, T cell tolerance restricts the extent of Id-driven T-B collaboration. However, a T cell repertoire exists toward rare V region sequences that depend on somatic mutations or possibly N-region diversity (17-19). Thus, low-frequency autoreactive B cells that express uncommon Id could haphazardly encounter Id-specific T cells in peripheral lymphoid tissues, resulting in Id-driven T-B interaction and autoimmunity (6,7,11,13,14,16).

Id-driven T-B collaboration and autoimmunity has been studied in mice that are transgenic for both Id⁺ Ig L-chain and Id-specific T cell receptors (TCRs) (10,14). Surprisingly, T cell tolerance toward Id was not complete in such doubly transgenic mice. Thus, a minor population of Id-specific T cells escaped tolerization, expanded as mice aged, and provided Id-driven help to Id⁻ B cells. Such Id-driven T-B collaboration caused secretion of high levels of IgG antibodies and ultimately severe systemic autoimmunity, including inflammatory bowel disease, arthritis, and kidney and skin diseases (14). NF-κB, originally identified in B cells (20,21), is a central transcription factor in both innate and adaptive immune responses. NF-κB is activated by a plethora of pro-inflammatory cytokines, chemokines, adhesion molecules, and immunoregulatory mediators. Inappropriate regulation of NF-κB has been associated with a number of disorders including arthritis, asthma, and inflammatory bowel disease (20,22).

At least two NF-κB signaling pathways exist (20,21). The classical pathway is dependent on the inhibitor of kappa B kinase beta and is involved in cytokine signaling, eg, tumor necrosis factor (TNF)α, interleukin 1, or pathogen recognition (Toll-like receptors) in inflammatory responses and innate immunity. The classical pathway is also triggered by TCR and BCR signaling (20,21). The alternative pathway is dependent on inhibitor of kappa B kinase alpha and is mediated through the NF-κB family members RelB and p52. The alternative pathway is involved in the development, homeostasis, and activation of adaptive immunity through ligands such as LTβ and CD40L (20,21). Thus, NF-κB is involved in several pathways resulting in inflammation.

### SUMMARY OF THE INVENTION

The present invention relates to the production and use of transgenic mice for research and drug discovery, and in particular to triple transgenic TCR⁺Id⁺Luc⁺ mice. In some embodiments, the present invention provides transgenic mice comprising an Id⁺ L chain transgene, an Id-specific TCR transgene, and a reporter transgene comprising NF-κB responsive promoter elements operably linked to a reporter gene. In some embodiments, the reporter gene encodes a bioluminescent or fluorescent protein.

In some embodiments, the present invention provides methods for producing transgenic mice comprising an Id⁺ L chain transgene, an Id-specific TCR transgene, and a reporter transgene comprising an NF-κB responsive promoter elements operably linked to a reporter gene comprising crossing a mouse line comprising an Id⁺ L chain transgene and an Id-specific TCR transgene with a mouse line comprising a reporter transgene comprising an NF-κB responsive promoter element operably linked to a reporter gene.

In some embodiments, the present invention provides methods for drug discovery comprising providing at least one drug and a transgenic mouse as described above, treating said mouse with said drug, and assaying expression of said reporter gene. In some embodiments, the present invention provides methods for testing a substance comprising providing at least test substance and a transgenic mouse as described above, treating said mouse with said test substance, and assaying expression of said reporter gene. In some embodiments, the present invention provides methods for evaluating a candidate drug for treating autoimmune disease comprising providing at least one candidate drug and a transgenic mouse as described above, treating said mouse with said candidate drug, and assaying expression of said reporter gene. In some embodiments, the autoimmune disease is a skin disease, small intestine disease, kidney disease, large intestine disease, or arthritis. In some embodiments, the autoimmune disease is Chagas disease, Chronic obstructive pulmonary disease, Crohns Disease, Dermatomyositis, Diabetes mellitus type 1, Endometriosis, Goodpasture's syndrome, Graves' disease, Guillain-Barré syndrome (GBS), Hashimoto's disease, Hidradenitis suppurativa, Kawasaki disease, IgA nephropathy, Idiopathic thrombocytopenic purpura, Interstitial cystitis, Lupus erythematosus, Mixed Connective Tissue Disease, Morphea, Multiple sclerosis (MS), Myasthenia gravis, Narcolepsy, Neuromyotonia, Pemphigus vulgaris, Pernicious anaemia, Psoriasis, Psoriatic Arthritis, Polymyositis, Primary biliary cirrhosis, Rheumatoid arthritis, Schizophrenia, Scleroderma, Sjogren's syndrome, Stiff person syndrome, Temporal arteritis, Ulcerative Colitis, Vasculitis, Vitiligor, Wegener's granulomatosis, Celiac Disease and inflammatory bowel disease.

In some embodiments, the foregoing methods further comprise one or more of the following steps: identifying a candidate drug from said treatment of said mouse, testing said drugs in humans, and providing said drug to human patients.

In some embodiments, the present invention provides methods of assessing progression of an autoimmune disease comprising: providing a transgenic mouse comprising an Id⁺ L chain transgene, an Id-specific TCR transgene, and a reporter transgene comprising NF-κB responsive promoter elements operably linked to a reporter gene with an experimental treatment, and assaying expression of the reporter gene. In some embodiments, the mouse is exposed to an experimental treatment. Examples of experimental treatments include administration of a drug or chemical compound, such as a small molecule organic compound, administration of a test diet, administration of a dietary supplement, exposure to radiation, treatment with an exercise regime, and the like.

### DESCRIPTION OF THE FIGURES

FIG. 1. Weight, survival, and clinical signs in triply transgenic mice. A cohort of 13 TCR⁺Id⁻Luc⁺ triply transgenic mice and 8 Id⁺Luc⁺ transgenic control littermates were inspected for disease and weighed weekly. A: Weight (left) and survival (right) of mice. B: Cumulative incidences of skin, joint and inflammatory bowel disease.

FIGS. 2A-H. NF-κB bioluminescence precedes and correlates with disease. A: Photons emitted from ventral aspects of mice with increasing age. Left: a representative TCR⁺Id⁻Luc⁺ mouse investigated at the indicated time-points. Right: an Id⁺Luc⁺ control. B-D: Focal disease. A common light intensity bar for all panels in B-D is shown (right). B: Early detection of skin disease on the dorsal aspect of head and upper thorax. Ears and eyes are traced in some frames with a black line. C: A representative 20-week-old triply transgenic mouse with severe skin disease including scarring is shown. Focal areas detected by bioluminescence (left) and photo (right), have been numbered. D: Early signals from the hind paw of a mouse that later developed arthritis. Clinical arthritis was only evident starting from week 14 (asterisks). E: Photons from ventral organs after sacrifice and skin removal. The ventricle and kidney are abbreviated as Vent and Kidn. The surface trace of the colon in the TCR⁺Id⁺Luc⁺ mouse is indicated with black lines. F: Signals from isolated organs post dissection. G: a) A whole spleen from a triple transgenic mouse displaying a focus of high bioluminescence signals (detected by the IVIS100) is shown. b) Light emissions from cross sections of the same spleen with focus were detected by a microscope with an ultrasensitive CCD camera (see Materials and Methods). Light emissions can be seen from the white pulp (W, traced with a black line). c) Immunofluorescence of an area with intense signals (see arrow from b) stained for IgG (red), peanut agglutinin [PNA] (green), and nuclei (blue). An active immune response with PNA⁺IgG⁻ germinal centers (yellow), PNA⁺IgG⁻ germinal centers (green), and IgG⁺ B cells (red) can been seen. H: NF-κB -dependent bioluminescence in triple transgenic treated with L-012; note the colocalization of NF-κB activity and inflammatory (L-012) signals from area of the distal colon. The inset shows NF-κB -dependent light emission postdissection from the distal third of the colon.

FIGS. 3A-B. Statistical evaluation of NF-κB bioluminescence signals in triply transgenic mice. A: Ventral surfaces of TCR⁺Id⁺Luc⁺ and Id⁺Luc⁺ mice (n=13, 10 respectively) were visualized and the photons emitted from the indicated areas were measured. Note the different scales on the y axis. *P<0.05, Mann Whitney. B: Quantification of signals from organs of mice, P values (Mann Whitney) are indicated.

FIGS. 4A-B. NF-κB bioluminescence correlates with autoantibody levels. A: IgG ANA titers correlated with emitted photons from the ventral sides of 10-week-old triply transgenic mice. A linear regression analysis is shown. B: The presence of IgG skin autoantibodies correlated with bioluminescence from ears measured at the indicated time points. Triply transgenic mice were divided into those positive or negative for serum autoantibodies against skin antigens at any of the indicated time points. The number of autoantibodypositive mice (out of 13) at each time-point is indicated. *P<0.05, Mann Whitney.

FIGS. 5A-F. Luciferase protein expression was detected in T cells, B cells, and macrophages, and in inflamed tissues. A-D: Representative examples of immunofluorescence analyses of 15 to 19-week-old mice are shown. A: Lymph node B cell follicles of Id⁺Luc⁺ (control) and TCR⁺Id⁺Luc⁺ mice were stained for luciferase protein (green), Id (anti-⁺2/3 2B6 mAb, red), and nuclei (DAPI, blue). NF-κB -activated Id⁺ B cells are yellow. B: Left: Interfollicular area showing Id-specific T cells (TCR-clonotype specific mAb GB113, red), and luciferase (green), and nuclei (DAPI, blue). An NF-κB activated Id-specific T cell (yellow), two non-activated Id-specific cells (red), as well as two NF-κB activated non-Id-specific T cells (green) are seen (T cells expressing exclusively endogenous TCRα-chains are not Id-specific and do not stain with GB11314). Right: interfollicular areas from Id⁺Luc⁺ (control) and TCR⁺Id⁺Luc⁻ mice showing CD11b (red) and luciferase (green) and nuclei (DAPI, blue). NF-κB CD11b⁺ macrophages are yellow. C: Triple staining of lymph node follicle showing Id⁺ B cells (anti-λ2/3 2B6 mAb, blue), Id-specific T cells (TCR-clonotype specific mAb GB113, red), and luciferase (green). The inset shows magnification and splitting of channels (right). Several Id⁺ B cells and Id-specific T cells cluster together, and both cell types are luciferase protein positive. D: Sections of an inflamed colon from a TCR⁺Id⁺Luc⁻ mouse. Left top: High magnification (×400) of area near erosion stained for Id (red), luciferase (green), and nuclei (blue). Id⁺ NF-κB⁺ B cells are yellow. Left, bottom: Co-localization of IgG (red) and C3 (green) at sites of erosion (asterisks). A cellular infiltrate is seen in the middle left of the panel. Right, low magnification (×100): luciferase (red), and complement C3 (green), nuclei (blue). The colon architecture is effaced by cellular infiltrates and erosions (asterisks). Areas where C3 and luciferase expression co-localize are yellow. E-F: Small intestines of mice with inflammation. E: H&E stain. Left, mononuclear cells infiltrating a villus (arrow). Right, shortening of villi. F: Staining of luciferase (red) and C3 (green) in triply transgenic (left) and doubly transgenic control mice (right).

FIGS. 6A-C. Antibodies related to intestinal pathology correlate with signals from inflamed small intestines. Sera were screened for antibodies associated with small intestinal pathology. A: Levels of serum anti-transglutaminase 2 (TG2) autoantibodies (left) and anti-Gliadin (right) correlated with NF-κB bioluminescence from small intestines isolated from TCR⁺Id⁺Luc⁺ mice. B: Serum from a 15-week-old triply transgenic mouse with small intestinal light emissions gave an anti-endomysium staining pattern, [IgG (red), and smooth muscle cell nuclei (blue)]. C: Autoantibodies directed toward the small intestine of a BALB/c RAG2−/− mouse. Left: serum from triply transgenic mouse (15 weeks old) with small intestinal signal and disease. Right: negative stain, serum from triply transgenic littermate (15 weeks) without small intestinal disease.

FIG. 7. λ2³¹⁵ Ig V cDNA and amino acid sequences.

FIG. 8. 4B2A1 TCR V cDNA and amino acid sequences (Vα and Vβ).

FIG. 9. Sequence for NF-kB responsive promoter.

### DEFINITIONS

To facilitate understanding of the invention, a number of terms are defined below.

The “non-human animals” of the invention comprise any non-human animal capable of expressing an Id⁺ L chain transgene, an Id-specific TCR transgene, and a reporter transgene comprising an NF-κB responsive promoter elements operably linked to a reporter gene. Such non-human animals include vertebrates such as rodents, non-human primates, ovines, bovines, ruminants, lagomorphs, porcines, caprines, equines, canines, felines, ayes, etc. Preferred non-human animals are selected from porcines (e.g., pigs), murines (e.g., rats and mice), most preferably mice and lagomorphs (e.g., rabbits).

The “non-human animals having a genetically engineered genotype” of the invention are preferably produced by experimental manipulation of the genome of the germline of the non-human animal at some point in time. These genetically engineered non-human animals may be produced by several methods including the introduction of a “transgene” comprising nucleic acid (usually DNA) into an embryonal target cell or integration into a chromosome of the somatic and/or germ line cells of a non-human animal by way of human intervention, such as by the methods described herein. Non-human animals which contain a transgene are referred to as “transgenic non-human animals”. A transgeneic animal is an animal whose genome has been altered by the introduction of a transgene.

The term “transgene” as used herein refers to a foreign gene that is placed into an organism, for example, by introducing the foreign gene into newly fertilized eggs or early embryos. The term “foreign gene” refers to any nucleic acid (e.g., gene sequence) which is introduced into the genome of an animal by experimental manipulations and may include gene sequences found in that animal so long as the introduced gene does not reside in the same location as does the naturally-occurring gene.

Embryonal cells at various developmental stages can be used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter which allows reproducible injection of 1-2 picoliters (pl) of DNA solution. The use of zygotes as a target for gene transfer has a major advantage in that in most cases the injected DNA will be incorporated into the host genome before the first cleavage [Brinster, et al. (1985) Proc. Natl. Acad. Sci. USA 82:4438-4442]. As a consequence, all cells of the transgenic non-human animal will carry the incorporated transgene. This will in general also be reflected in the efficient transmission of the transgene to offspring of the founder since 50% of the germ cells will harbor the transgene. Micro-injection of zygotes is the preferred method for incorporating transgenes in practicing the invention.

U.S. Pat. No. 4,873,191 describes a method for the micro-injection of zygotes; the disclosure of this patent is incorporated herein in its entirety.

Retroviral infection can also be used to introduce transgenes into a non-human animal. The developing non-human embryo can be cultured in vitro to the blastocyst stage. During this time, the blastomeres can be targets for retroviral infection [Janenich (1976) Proc. Natl. Acad. Sci. USA 73:1260-1264]. Efficient infection of the blastomeres is obtained by enzymatic treatment to remove the zona pellucida [Hogan et al. (1986) in Manipulating the Mouse Embryo, Cold Spring Harbor Laboratory Press, Cold Spring Harbor, N.Y.]. The viral vector system used to introduce the transgene is typically a replication-defective retrovirus carrying the transgene [Jahner, D. et al. (1985) Proc. Natl. Acad Sci. USA 82:6927-6931; Van der Putten, et al. (1985) Proc. Natl. Acad Sci. USA 82:6148-6152]. Transfection is easily and efficiently obtained by culturing the blastomeres on a monolayer of virus-producing cells [Van der Putten, supra; Stewart, et al. (1987) EMBO J. 6:383-388]. Alternatively, infection can be performed at a later stage. Virus or virus-producing cells can be injected into the blastocoele [Jahner, D. et al. (1982) Nature 298:623-628]. Most of the founders will be mosaic for the transgene since incorporation occurs only in a subset of cells which form the transgenic animal. Further, the founder may contain various retroviral insertions of the transgene at different positions in the genome which generally will segregate in the offspring. In addition, it is also possible to introduce transgenes into the germline, albeit with low efficiency, by intrauterine retroviral infection of the midgestation embryo [Jahner, D. et al. (1982) supra]. Additional means of using retroviruses or retroviral vectors to create transgenic animals known to the art involves the micro-injection of retroviral particles or mitomycin C-treated cells producing retrovirus into the perivitelline space of fertilized eggs or early embryos [PCT International Application WO 90/08832 (1990) and Haskell and Bowen (1995) Mol. Reprod. Dev. 40:386].

A third type of target cell for transgene introduction is the embryonal stem (ES) cell. ES cells are obtained by culturing pre-implantation embryos in vitro under appropriate conditions [Evans, et al. (1981) Nature 292:154-156; Bradley, et al. (1984) Nature 309:255-258; Gossler, et al. (1986) Proc. Acad. Sci. USA 83:9065-9069; and Robertson, et al. (1986) Nature 322:445-448]. Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal. For review see Jaenisch, (1988) Science 240:1468-1474. Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells which have integrated the transgene. This technique obviates the need for growth of the transfected ES cells under appropriate selective conditions prior to transfer into the blastocoel.

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The terms “promoter element” or “promoter” as used herein refers to a DNA sequence that precedes a gene in a DNA polymer and provides a site for initiation of the transcription of the gene into mRNA.

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

The term “reporter gene” refers to a nucleic acid sequence which encodes a product that is easily and quantifiably detected. Reporter genes are often operably linked to known or putative promoter and/or enhancer elements to permit the study of these elements (i.e., to define regions upstream of genes which are responsible for tissue-specific expression and/or constitutive or basal levels of expression). Genes commonly used as reporter genes in the study of eukaryotic gene expression include the bacterial genes encoding β-galactosidase, chloramphenicol acetyltransferase and β-glucuronidase.

### DETAILED DESCRIPTION OF THE INVENTION

To monitor NF-κB-associated inflammation in mice undergoing Id-driven autoimmune disease, the present invention provides triple transgenic mice expressing both Id⁺ B cells and Id-specific CD4⁺ T cells (14) as well as a luciferase reporter transgene under NF-κB control (23). On NF-κB activation and nuclear translocation of NF-κB, luciferase activity could be repeatedly monitored in intact mice by whole body bioluminescence imaging. Moreover, results were corroborated at the cellular level by detection of luciferase protein expression in single cells. The results show that imaging of NF-κB activation permits early detection of subclinical disease as well as tracking of disease development.

It is desirable to have an early and sensitive detection marker of autoimmune disease in intact animals. Nuclear factor (NF)-κB is a transcription factor that is associated with inflammatory responses and immune disorders. Previously, the inventors demonstrated that so-called idiotypic-driven T-B cell collaboration in mice doubly transgenic for paired immunoglobulin and T cell receptor transgenes resulted in a systemic autoimmune disease with systemic lupus erythematosus-like features. The present invention allows investigation of NF-κB activation by including an NF-κB-responsive luciferase reporter transgene in this animal model. Triply transgenic mice developed bioluminescence signals from diseased organs before onset of clinical symptoms and autoantibody production, and light emissions correlated with disease progression. Signals were obtained from secondary lymphoid organs, inflamed intestines, skin lesions, and arthritic joints. Moreover, bioluminescence imaging and immunohistochemistry demonstrated that a minority of mice suffered from an autoimmune disease of the small intestine, in which light emissions correlated with antibodies against tissue transglutaminase and gliadin. Detection of luciferase by immunohistochemistry revealed NF-κB activation in collaborating B and T cells, as well as in macrophages. These results demonstrate that bioluminescent in vivo imaging of NF-κB activation can be used for early and sensitive detection of autoimmune disease in an experimental mouse model, offering new possibilities for the evaluation of anti-inflammatory drugs. (Am J Pathol 2009, 174:1358-1367; DOI: 10.2353/ajpath.2009.080700).

In some embodiments, the present invention provides transgenic mice produced from a cross between two different transgenic mouse models: 1) Double transgenic TCR/Id-autoimmune disease model, and 2) NF-κB luciferase reporter model. Model 1 contains a modified T-cell receptor and a B-cell receptor (antibody). The T-cell receptor interacts specifically with fragments of the B-cell receptor (Id), presented on MHC II, resulting in a stimulation of the B-cell. In some cases these B-cells will express auto-antibodies and still be activated by the T-cell specific activation. The result is that these mice produce auto-antibodies and develop a plethora of auto-immune disease. Model 2 consists of NF-κB binding sites coupled to luciferase as a reporter gene. Thus the expression of luciferase (and the NF-κB activity), can be monitored in both living animals and in excised tissues. In preferred embodiments, non-invasive monitoring is particularly appealing since the regulation of NF- KB during disease progression can be tracked over a long period of time. In some embodiments, the transgenic mice are used to track autoimmune disease using NF-κB as a marker of disease and inflammation. In some embodiments, the transgenic mice express a heterologous TCR comprising alpha and beta variable regions that are at least 80%, 90%, 95% or 100% identical to SEQ ID NOs:4 and 6 (amino acid sequences) or SEQ ID NOs:3 and 5 (cDNA sequences). In some embodiments, the transgenic mice express a heterologous immunoglobulin comprising a V gene sequence that is at least 80%, 90%, 95% or 100% identical to SEQ ID NO:2 (amino acid sequence) or SEQ ID NO:1 (cDNA sequence). In some embodiments, the mice comprise a heterologous transgene comprising a NF-κB responsive promoter that is at least 80%, 90%, 95% or 100% identical to SEQ ID NO:9.

The transgenic animals of the present invention find use in a variety of applications, including, but not limited to, identification of biochemical pathways implicated in a variety of diseases, especially autoimmune diseases, and in drug screening applications. For example, in some embodiments, test compounds (e.g., compounds suspected of alleviating or altering the symptoms of an autoimmune disease) and control compounds (e.g., a placebo) are administered to the transgenic animals and the control animals. The effects of the test and control compounds on the transgenic animal or disease symptoms are then assessed. In still further embodiments, the expression (e.g., overexpression or underexpression) of the reporter gene in a particular tissue of interest is examined.

In some embodiments, the present invention provides methods for examining the progress or the effect of autoimmune diseases in the presence or absence of an experimental treatment. Examples of autoimmune diseases include, but are not limited to, inflammatory bowel disease, skin disease, psoriasis, arthritis, rheumatoid arthritis, vasculitis, kidney disease, nephritis, celiac disease, dermatitis, atopic dermatitis, cardiomyopathy, Crohn's disease, colitis, lupus, exzema, fibromyalgia, Grave's disease, Guillian-Barre syndrome, glomerulonephritis, multiple myeloma, multiple sclerosis, and the like. In some embodiments, the experimental treatment is application of test compound or substance to the mouse. Such test substances and compounds can be delivered by any appropriate route, including, but not limited to, the following routes: oral, topical, rectal, intranasal, intramuscular, subcutaneous, intravitreal, parenteral or enteral. In some embodiments, the test treatment may be for example, administration of a test diet, a dietary supplement, a physical treatment, exercise, electrostimulus, radiation, and the like. In some embodiments, the methods of the present invention further comprise analyzing the transgenic mouse for autoimmune disease progression or effect. In some embodiments, the analysis comprises analyzing the mouse for expression of the reporter gene. In some embodiments, the whole mouse is analyzed for reporter gene expression, preferably by whole body bioluminescence imaging. In some embodiments, specific tissues or organs are excised and analyzed for reporter gene expression. In some embodiments, the mouse, tissues or organs are sectioned prior to analysis of reporter gene expression. In some embodiments, where the product of the reporter gene reacts with a substrate, the substrate is applied to the mouse, tissue or organ and an assay is conducted. In some preferred embodiments, the reporter gene is luciferase and the substrate is a substrate that is acted upon by the luciferase such as luciferin. In some embodiments, the assay comprises detection of bioluminescence.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994])); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nat'l. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

The present invention further provides agents identified by the above-described screening assays. Accordingly, it is within the scope of this invention to further use an agent identified as described herein in an appropriate animal model (such as those described herein) to determine the efficacy, toxicity, side effects, or mechanism of action, of treatment with such an agent. Furthermore, agents identified by the above-described screening assays can be used for treatment of chronic and hereditary autoimmune diseases.

Experimental

Materials and Methods

Mice

The hemizygous λ2³¹⁵ Ig L-chain transgenic mice [Id⁺-mice] (24) and the hemizygous αβ TCR transgenic mice (25) specific for the Id(λ2³¹⁵)-peptide (amino acids 91 to 101) presented on I-E^{d} are both on a BALB/c background and have been made homozygous for their transgenes (Id⁺/⁺ and TCR⁺/⁺ respectively). Mice transgenic for a luciferase (Luc) reporter construct under control of NF-κB responsive elements and with a CBAxC57BL/6 background were back-crossed three times with BALB/c mice. Offspring that were typed as NF-κB-Luc (Luc⁺/⁻) were crossed with homozygous TCR transgenic mice (TCR⁺/⁺) on a BALB/c background. Resulting litters were typed for MHC haplotype and Luc. Offspring that were TCR⁺/⁻Luc⁺/⁻H2^{d/d} were further crossed with homozygous Id⁻/⁺ mice (BALB/c background), resulting in litters composed of TCR⁺/⁻ Id⁺/⁻Luc⁺/⁻ experimental mice and TCR⁻/⁻Id⁺/⁻Luc⁺/⁻ control mice. All mice were thus H2^{d/d} but had considerable contributions of CBA and C57BL/6 to a predominantly BALB/c background. TCR⁺/⁻Id⁻/⁻Luc⁺/⁻ control mice were H2^{d/d} homozygous offspring from an independent (TCR⁺/⁺ X Luc⁺/⁻) cross. Mice were typed for expression of Id⁺ L-chain and TCR transgenes by flow cytometry and for the NF-κB transgene by injection of luciferin substrate and measurement of in vivo bioluminescence.

Clinical Assessment of Mice

Mice were weighed weekly Skin disease was scored as positive when skin was clearly inflamed with induration, erythema, loss of hair, scabbing, and scarring. Arthritis entailed swollen joints and redness of overlying skin. Mice with perianal erythema, rectal prolapse, diarrhea, and mucoid/bloody stool were defined as having inflammatory bowel disease.

Enzyme-Linked Immunosorbent Assay Hamster tissue transglutimase 2 (TG2) and trypsin digested Gliadin, kindly provided by Dr L. Sollid (Oslo), was coated onto 96-well plates, diluted sera was added, and plates were developed by biotinylated rat-anti IgG monoclonal antibody (mAb, BD Pharmingen) essentially as described (14).

Measurements of Serum Immunoglobulins and Autoantibodies

Immunoglobulins were detected by enzyme-linked immunosorbant assay as described (14). Diluted sera were added to HEp2 cells (Immuno Concepts) and antinuclear antibodies (ANA) were detected with Alexa Fluor 488 goat anti-IgG2a and Alexa Fluor 546 goat anti-IgG1 (Molecular probes).

Immunohistochemistry

Organs were embedded in optimal cutting temperature compound, and 5-μm frozen sections were mounted on L-polylysine-coated glass slides, air-dried overnight, and blocked with 30% heat-aggregated rat serum. All further solutions contained 30% rat serum. Double staining: Slides were stained with Rabbit anti-luciferase (Sigma-Aldrich St. Louis, Mo.) in PBS w/30% rat serum. Nuclei were stained with 4,6-diamino-2-phenylindoldi-hydrochloride (DAPI, Molecular Probes, Eugene, Oreg.). Slides were further developed with highly cross-absorbed goat anti-rabbit (H+L)-Alexa 488 (Molecular Probes), followed by signal amplification with donkey anti-goat-Alexa 488 (Molecular Probes). Fluorescein isothiocyanate-conjugated F(ab′)2 goat-anti-mouse-C3 was from Cappel ICN, Costa Mesa, Calif. The following mAbs were biotinylated in our lab: transgenic TCR-clonotype-specific GB113,26 anti-CD4 (GK1.5), anti-CD11b (TIB198), anti-MHC class II (TIB120), and 2B6 (anti-λ2/3)(24). MAbs were purified and biotinylated in our lab and were detected with streptavidin-Cy3 from Amersham Pharmacia Biotech (Piscataway, N.J.). Triple staining: Luciferase was stained as described above. In addition, biotinylated 2B6 (anti-λ2/3), developed with streptavidin-Alexa 355 (Molecular Probes), was used for detection of Id⁺ B cells. Biotinylated GB113, developed with streptavidin Cy3, was used for detection of Id-specific T cells. Biotin was blocked by avidin (Sigma-Aldrich) 10 μg/ml, followed by addition of biotin (Sigma-Aldrich). Luciferase-staining could also be detected in acetone or ethanol-fixed slides.

Imaging and Luciferase Measurements

Cohorts of littermates were investigated for NF-κB activity with a luciferin-based bioluminescence assay, from three weeks of age. Isoflurane (2.5%) was used as anesthetic to immobilize the mice, followed by an i.p injection of D-luciferin (120 mg/kg; Biothema, Dalaro, Sweden), dissolved in 200 μl PBS pH 7.8. Immediately afterward, the mice were placed in a light-sealed chamber connected to a sensitive and cooled CCD camera (IVIS-100 Imaging System, Xenogen). Luminescence emitted from the mouse was integrated for 1 minute starting 8 minutes after the injection of luciferin. In some experiments, mice were sacrificed; individual organs were excised from the mice 10 minutes after D-luciferin injection, and imaged. Light emission was analyzed on the Living Image 2.0 software and expressed as photons/sec/cm²/steradian. Organ homogenates were made by homogenizing frozen tissues in lysis buffer (Promega, Madison, Wis.). The luciferase activity in homogenates was assessed as described previously.23 The luminescent probe L-012 (8-amino-5-chloro-7-phenylpyrido[3,4-day]pyridazine-1,4-(2H,3H)dione),27 WakoPure Chemical Industries (Osaka, Japan) emits light in presence of oxygen radicals. L-012 was injected i.p. (25 mg/kg in 100 μl ddH2O), and mice were imaged 10 to 15 minutes later in the IVIS-100 Imaging system as described above. In some experiments a microscope with an ultrasensitive CCD camera (Andor iXon DV887, Andor Technology, Belfast, Northern Ireland) was used to image emitted light from sections.

Statistics

The Mann-Whitney test was used throughout the paper to calculate the indicated P values.

Results

Neither Introduction of NF-κB-Luciferase Reporter Transgenes Nor Altered Genetic Background Affect the Course of Autoimmune Disease Caused by Chronic Id-Driven T-B Collaboration

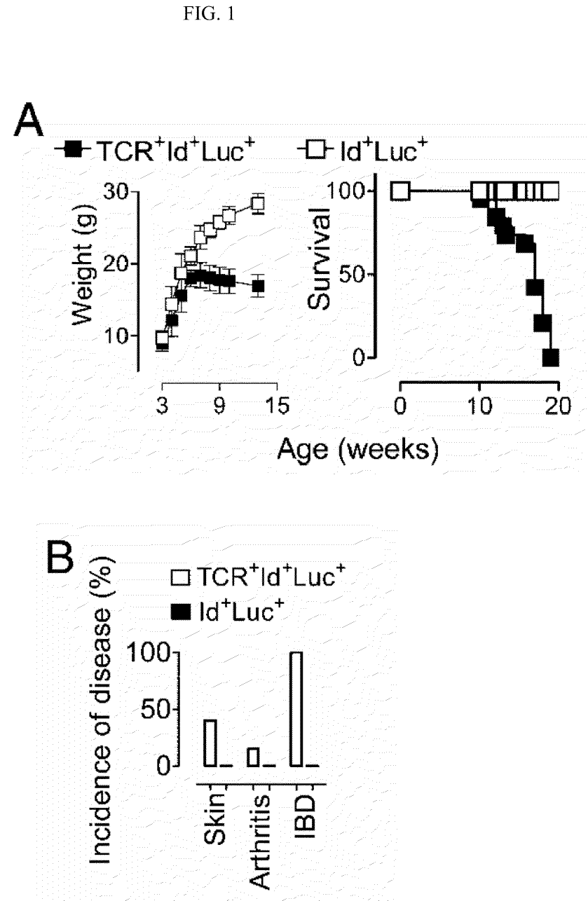

The aim of the present study was to see if NF-κB activation could serve as an early in vivo marker for detection of autoimmune disease elicited by chronic Id-driven T-B collaboration. To this end we bred NF-κB-luciferase reporter transgenes into doubly Id-specific TCR/Id⁺Ig transgenic mice previously described to develop hypergammaglobulinemia, IgG autoantibodies and lethal SLE-like disease (14). A potential caveat to this approach is that the multiple NF-κB responsive elements could absorb NF-κB and thereby thwart disease development in triply (TCR ⁺Id⁺NF-κB-Luc⁺) transgenic mice. Another concern was that in-breeding of the NF-κB-Luc transgenes resulted in triply transgenic mice with substantial amounts of CBA and C57Bl/6 background genes, which could alter the autoimmune disease previously observed in doubly transgenic mice on a pure BALB/c background (14). However, triply transgenic mice developed lethal disease (FIG. 1A) with clinical signs (FIG. 1B) and development of IgG autoantibodies (see below) that did not differ significantly from that previously observed in doubly TCR⁻Id⁺ transgenic mice (14). Thus, CBA and C57BL/6 background genes did not markedly influence disease development, although minor effects cannot be ruled out. Both TCR and Id⁺ L chain transgenes were needed for disease development since neither TCR⁺ NF-κB-Luc⁺ nor Id⁺ NF-κB-Luc⁺ doubly transgenic mice developed disease (FIG. 1 and data not shown).

Measurement of Luciferase Activity

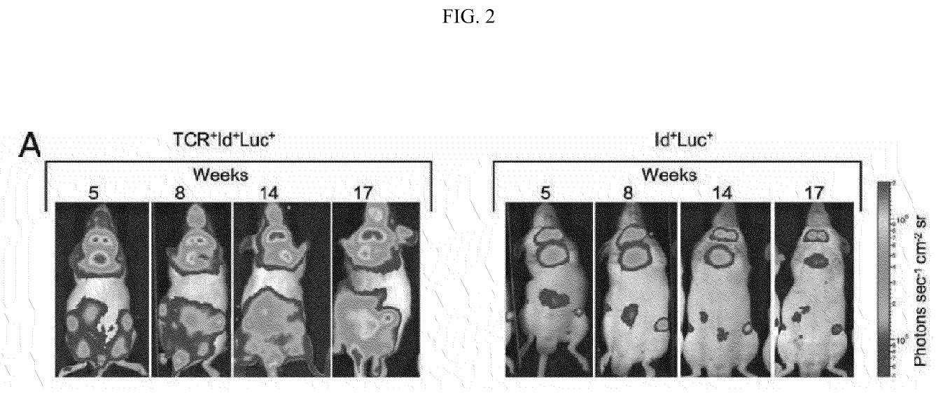

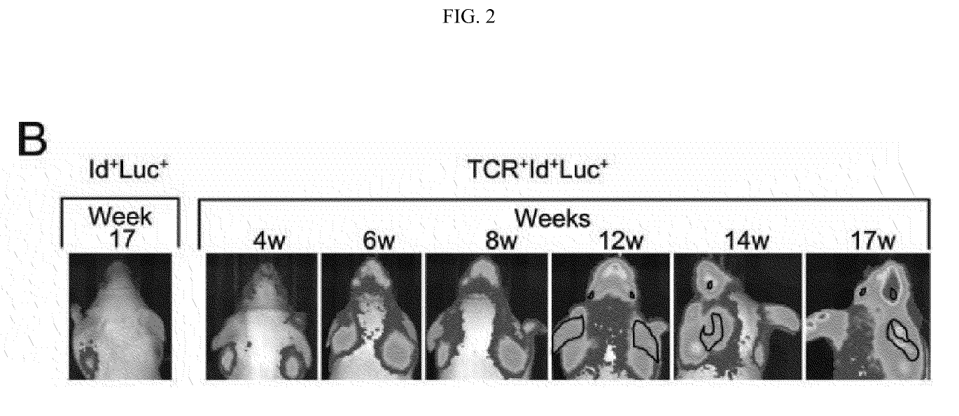

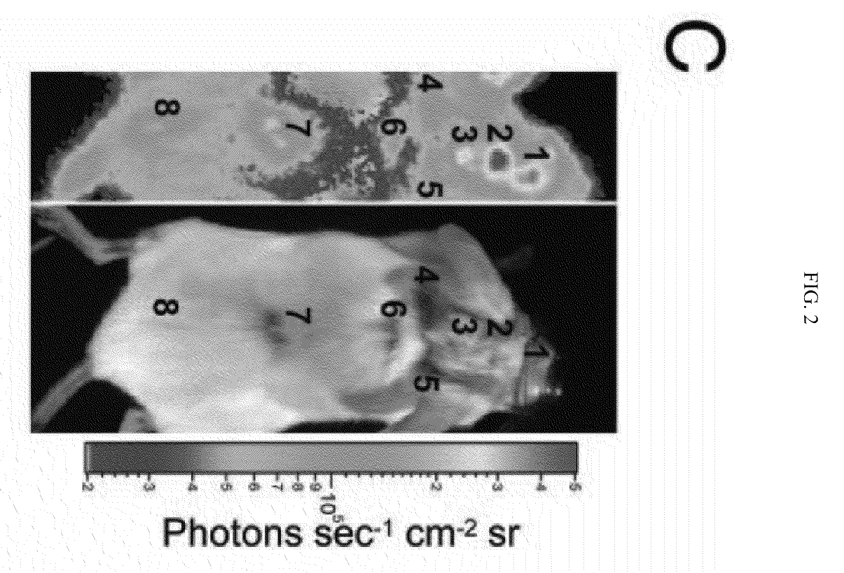

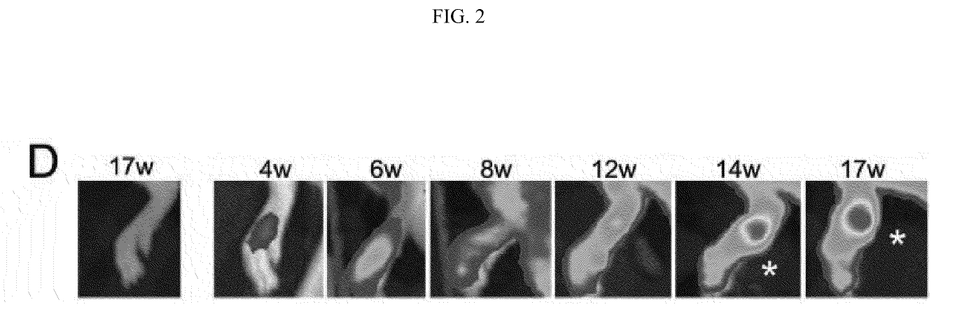

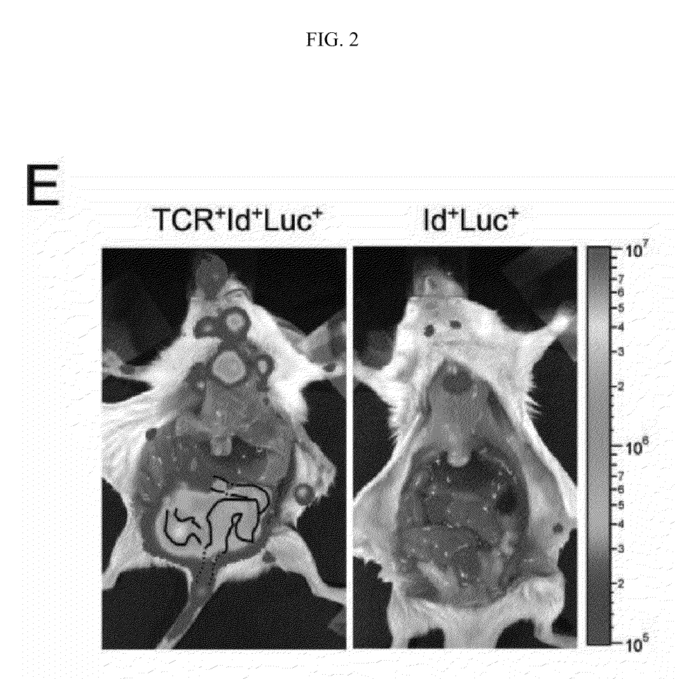

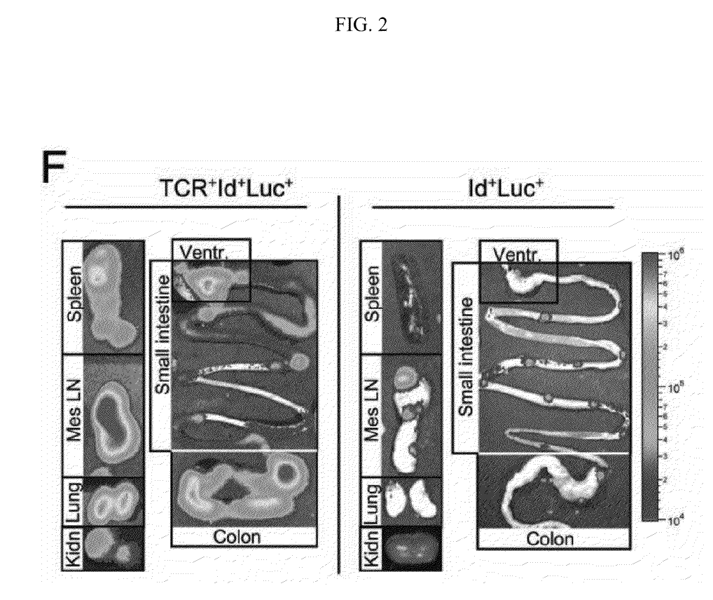

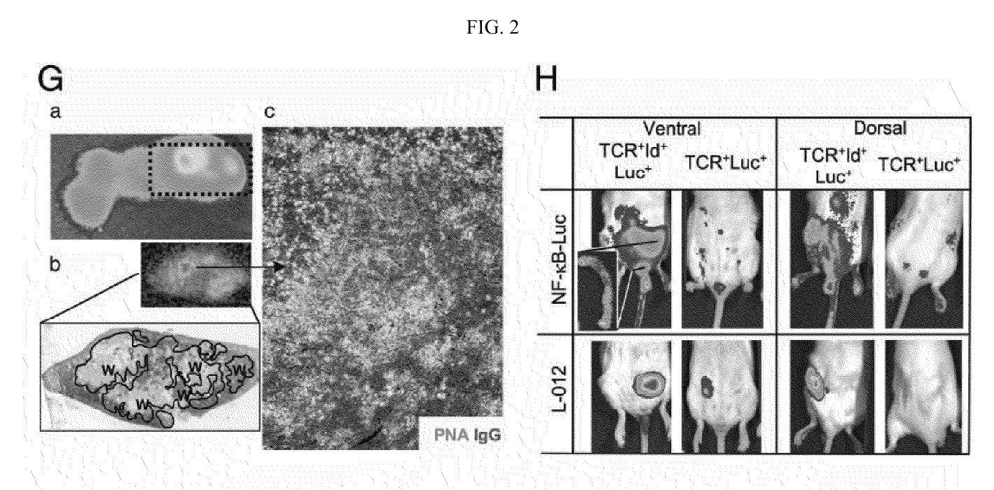

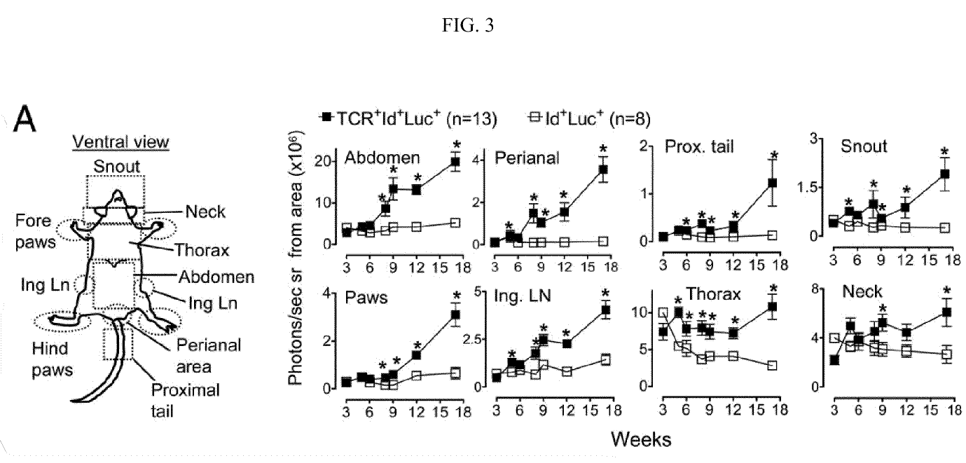

Triply transgenic mice were injected with the substrate luciferin and photon emissions were externally detected by use of an IVIS 100 apparatus (Xenogen) (FIG. 2A-D). Mice that lacked either TCR or Ig L chain transgenes (ie, Id⁺ NF-κB-Luc⁺and TCR⁺ NF-κB-Luc⁺ mice) were used as negative controls; for reasons of simplicity, both these controls are not always shown in the results given below. In some experiments, mice were sacrificed and imaged either after removal of skin (eg, FIG. 2E), or after explantation of organs (eg, FIGS. 2F and 3B). The luciferase activity in organ homogenates (23) gave similar results (data not shown). For closer scrutiny of luciferase activity within an organ, luciferin was sometimes dropped onto sections and light observed with an ultrasensitive CCD camera (eg, FIG. 2G). To differentiate the cellular origin of the luciferase signal, a staining method was developed that revealed luciferase protein by immunohistochemistry. Using these different multilevel approaches, we below describe organ involvements and cellular origins of NF-κB-dependent light signals in triply transgenic mice suffering from Id-driven autoimmune disease.

Early Detection of Increased NF-κB Activity in Living Triply Transgenic Mice

Whole animals were repeatedly imaged for several months starting from week 3. Ventral views gave the brightest signals, presumably because such a view allows detection of photons emanating from internal organs (see below). From week 5, triply transgenic mice had a detectable and progressive increase in bioluminescence signals compared with Id⁺ Luc⁺ mice (FIG. 2A). Emission intensities were slightly lower on some days presumably due to luciferin substrate variability, but the anatomical foci and ratio (triply transgenic versus control) of light emissions were robust and reproducible. More specifically, the signals were strongest in the abdominal area, but significant and progressively increasing signals were also obtained from the neck, the thorax, the snout, the perianal region, the proximal tail, the paws, and the inguinal lymph nodes (FIG. 3A). The increased signals required both Id⁺ transgenes and TCR transgenes because both Id⁺ Luc⁺ mice (FIGS. 2A and 3A) and TCR⁺Luc⁺ mice (not shown) had only low photon emissions.

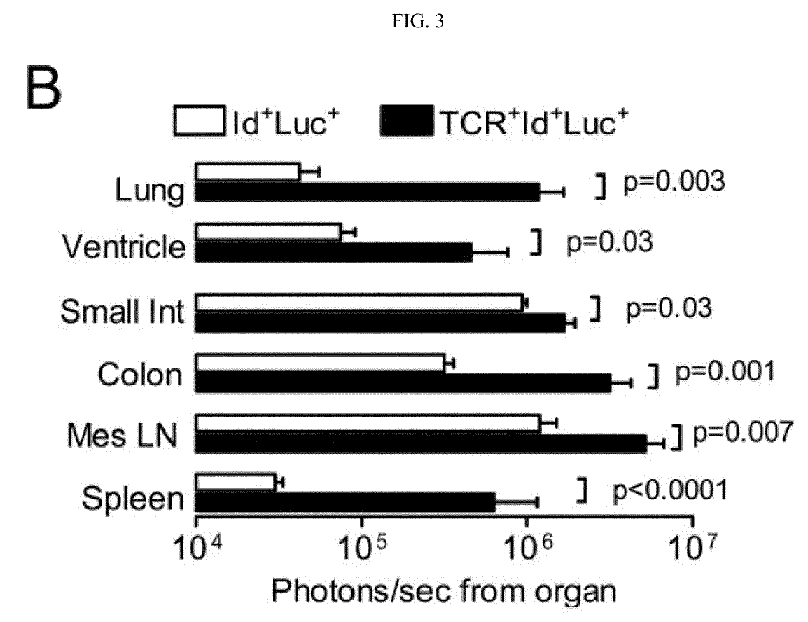

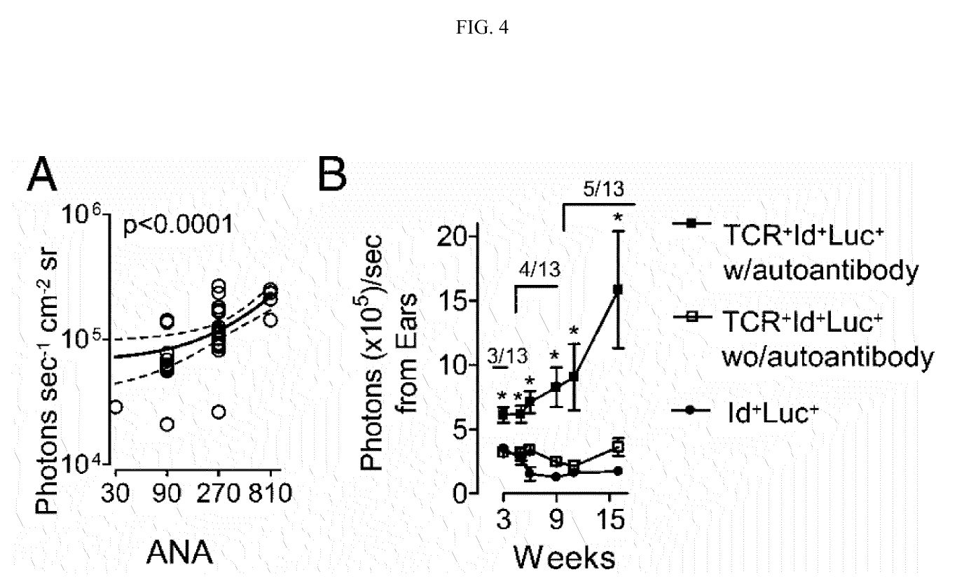

Importantly, the increased bioluminescence signals in triply transgenic mice started approximately at the same time that mice failed to gain weight (FIG. 1A), and several weeks before organ-specific clinical signs of disease (see below). In agreement with previous studies in doubly transgenic mice, 14 triply transgenic mice had detectable antinuclear IgG serum antibodies first from week 8, which is several weeks delayed compared with the increase in ventral bioluminescence signals observed in intact mice. Sera exhibited a homogenous ANA staining pattern (14) with antibodies predominantly of an anti-dsDNA specificity (L. M., B. B., manuscript in preparation). No IgG autoantibodies were detected in Id⁺ Luc⁺ controls. ANA titers increased with age of triply transgenic mice (data not shown). When investigated by 10 weeks of age, ANA titers correlated with ventral light emissions (FIG. 4A). With advancing age, the ANA-titers as well as ventral bioluminescence signals increased, both parameters rising about twofold by week 14 (data not shown).

Early Detection of Bioluminescence Signals from Lymphoid Organs

Doubly TCR⁻Id⁺ transgenic mice have a pronounced deletion of Id-specific thymocytes due to presentation of Id-peptide/class II molecules in the thymus (10,14). Interestingly, the triply transgenic mice had increased signals from areas corresponding to the thymus, suggesting that negative selection of thymocytes might be associated with activation of NF-κB (FIG. 2, A and E).

We have previously described that a few Id-specific T cells escape negative selection in doubly transgenic mice and that these escapees with time expand in the periphery and cause chronic Id-driven T-B collaboration and autoimmune pathology (14). Consistent with this, triply transgenic mice developed increased bioluminescence in cervical and inguinal lymph nodes from 5 weeks of age (FIG. 2, A and E). The signals gradually increased with time, suggesting an intensification of Id-driven immune responses with age (FIG. 3A). Explanted esenteric lymph nodes and spleens had strongly increased signals (FIGS. 2F and 3B). Moreover, foci of intense activity could be detected on spleen sections exposed to luciferin. The activity co-localized with the white pulp (FIG. 2G).

Id⁺ B Cells and Id-Specific T Cells in Synapse Both Express Luciferase Protein

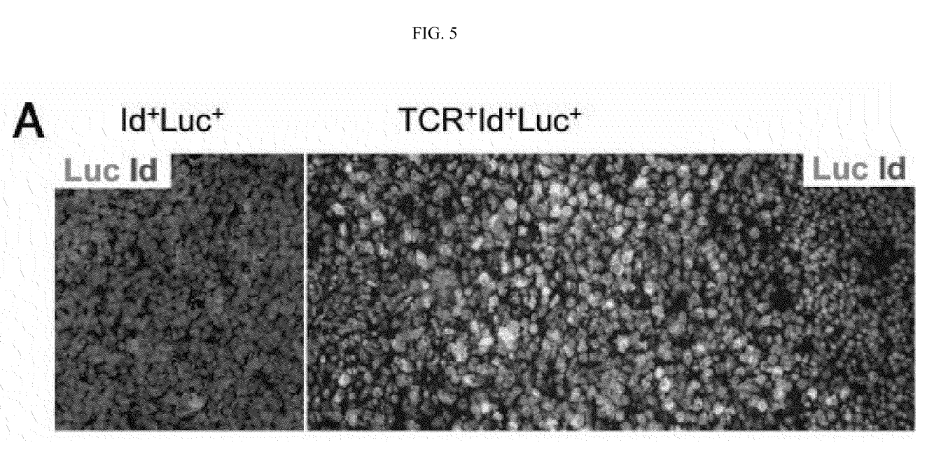

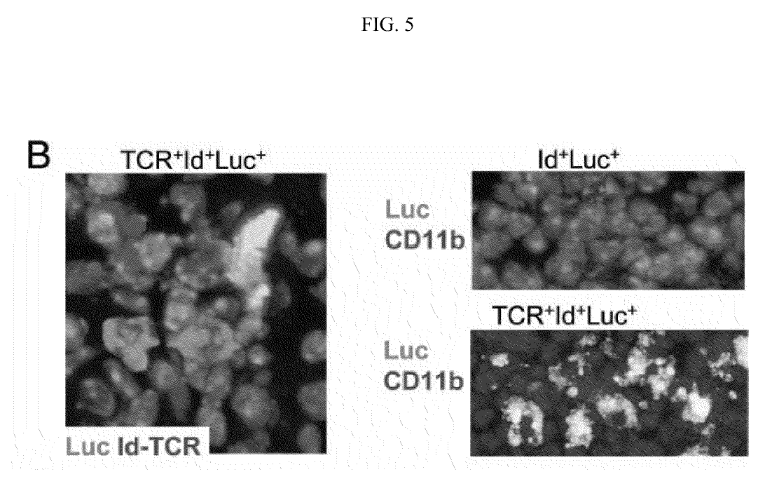

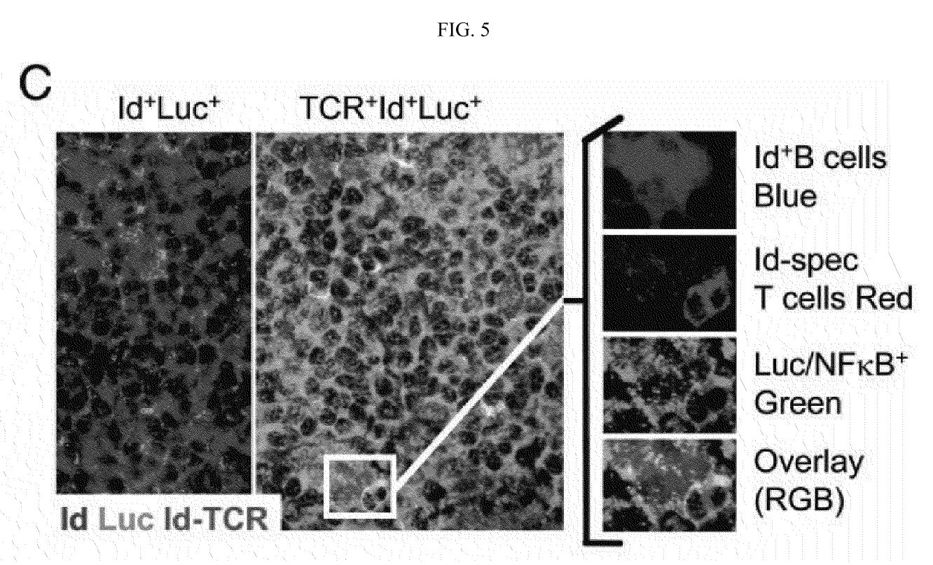

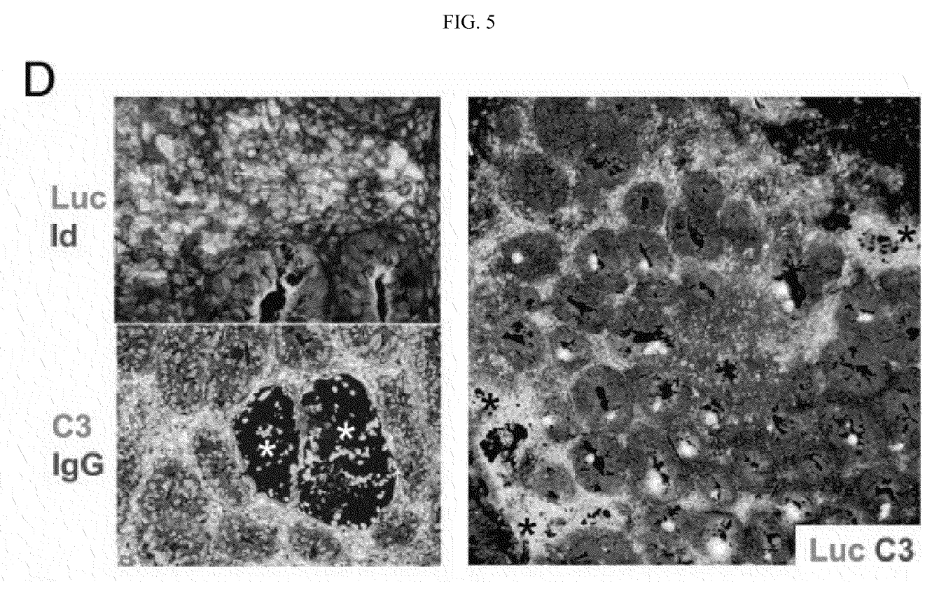

To determine exactly which cells were responsible for such bioluminescence, we developed a staining method for detection of luciferase protein expressed by NF-κB activated cells. Id⁺ B cells in lymph nodes and spleens of triply transgenic mice frequently stained positive for luciferaseprotein, both in follicular and extra-follicular areas (FIG. 5A and data not shown). Id-specific T cells also expressed luciferase protein (FIG. 5B), as did extra-follicular CD11b⁺ macrophages (FIG. 5B).

It has previously been shown that Id-specific T cells activate Id⁺ B cells by cognate Id-driven collaboration (13,14). To investigate whether Id⁺ B cells and Id-specific T cells in close proximity both expressed NF-κB, we performed triple staining Indeed, clusters of Id-specific T cells and Id⁺ B cells were readily found, and both cell types had activated NF-κB (FIG. 5C).

To test whether activation of Id-specific T cells via their TCR was sufficient to stimulate NF-κB and light emissions in vivo, we transferred Id-specific Luc⁺ T cells into RAG2⁻/⁻ mice. To avoid background emissions due to homeostatic expansion of T cells in T cell-deficient hosts, we waited 60 days before transfer of either Id⁺ or Id⁻ splenocytes. After 24 hours, a fourfold increase of light emissions was seen after injection of Id⁺ cells, while no increase was observed in recipients of Id⁻ cells (L. M., H. C., & B. B., manuscript in preparation). The above findings done in vivo were corroborated by in vitro experiments where Id-specific Luc⁺ Th2 cell lines emitted light after exposure to Id⁻, but not Id⁻ B cells (data not shown).

Early NF-κB-Mediated Signals from the Large Intestine

Besides a systemic autoimmune disease with SLE-like features, chronic Id-driven T-B collaboration resulted in inflammatory bowel disease in all mice with massive accumulations of Id-specific T cells, Id⁺ B cells and macrophages in inflamed colons (14). Consistent with this, triply transgenic mice had a strongly increased signal from the lower part of the abdomen and the perianal area (FIGS. 2A and 3A). Importantly, light signals from these areas predated the appearance of mucoid bloody diarrhea, a sign of inflammatory bowel disease, by several weeks. On opening of the abdominal cavity, signal patterns demarking the large bowel could be seen (FIG. 2E).

Sections revealed transmural inflammation with multiple areas of erosions. Luciferin protein co-localized to areas with inflammation, complement/IgG immune complex deposition and cellular infiltrates, and luciferase activity was readily detected in both Id⁺ B cells and other cells yet to be defined (FIG. 5D). Notably, some mice had bioluminescent stools, indicating shedding of NF-κB expressing cells (data not shown).

The L-012 probe emits photons when activated by reactive oxygen radicals and has previously been used to visualize degranulation of phagocytes in vitro (27). When triply transgenic mice were injected with this probe, a discrete signal was obtained that corresponded to an area of colon that was also revealed by injection of the luciferin substrate (FIG. 2H). The reason why the L-012 probe only gave a signal localized to the colon, and not other sites revealed by the luciferin substrate, could be related to the severe inflammation of the colon and infiltration by activated macrophages.

Early Detection of Vasculitis

We have previously observed that adult doubly transgenic mice often lost distal parts of their tails due to vasculitis (14). Consistent with this, proximal tail signals were in the present experiments significantly increased from week 6 (FIG. 3A), although no scarring or autoamputation due to vasculitic disease was seen before week 20.

Early Detection of Arthritis

Previously, we have described that about 20% of Id⁺ TCR⁺ mice develop arthritis characterized by pannus formation, cartilage destruction and bone erosion (14). Based on clinical inspection, 15% of the triply transgenic mice in the current study had arthritic disease with metacarpal/tarsal swelling and erythema (FIG. 1B). However, nearly half the mice had signals from one or more paws, and the increased signal was significant as early as from week 8 (FIG. 3A). In fact, mice that developed marked swelling of joints emitted photons 5 to 8 weeks before clinically obvious disease (FIG. 2D, and data not shown).

Early Detection of Skin Disease

Signals were also significantly augmented from the dorsal aspects of the mice, reflecting skin disease in the relative absence of signals from internal organs. From weeks 5 to 8, snout, periorbital, and ear signals were seen (FIG. 2B), antedating clinical skin and eye affection by several weeks (FIG. 2B). From week 12, marked luminescence signals were found in localized areas in some mice, suggesting skin disease (eg, from ears), although no visual signs of disease (except a few cases of mild erythema) were evident by mere inspection (FIG. 2B). From weeks 17 to 19, more intense signals appeared on the dorsal aspects of mice corresponding to sites of more severe disease (FIG. 2C). Interestingly, some foci of increased bioluminescence appeared normal by clinical inspection (FIG. 2C, see spot 8). In summary, only 5 of 13 mice had clinically obvious skin disease before 20 weeks of age, with inflammation and scarring (FIG. 1B). However, already at 6 weeks of age 8/13 had emissions from the facial skin, and by 20 weeks, 12/13 had bright skin signals. Areas of clinical skin disease always had an increased NF-κB signal. These results indicate that NF-κB signal from the skin is a sensitive and early indicator of skin inflammation.

Detection of Kidney Disease

We have previously described that more than a third of Id⁺ TCR⁺ mice have detectable IgG and C3 depositions in kidney glomeruli (14). However, the glomerulonephritis is relatively mild as compared with that seen in end-stage disease in BWF1 and MRL/lpr mice strains (28), and only very low level proteinuria has been seen (L. M., B. B., unpublished). In the present study, at 20 weeks of age, significantly increased but modest bioluminescence signals were obtained from the dorsal aspects overlying the kidneys of TCR⁺Id⁻Luc⁺ mice (see supplemental Figure S1 at http://ajp.amjpathol.org). Even so, it may be difficult ascribe such relatively weak light emissions to the kidney, because the skin or the perirenal tissue may contribute to the signal. However, on dissection, kidneys of triply transgenic mice exhibited enhanced bioluminescence (FIG. 2F).

NF-κB Signals Correlate with IgG Anti-Nuclear and Skin Autoantibodies

We have previously reported that doubly TCR⁺Id⁺ mice develop IgG autoantibodies that bind tissue antigens and activate complement at sites of disease (14). It was therefore of interest to test if levels of skin-specific autoantibodies (see supplemental Figure S2 at http://ajp.amjpathol.org) correlated significantly with bioluminescence signals from skin so readily accessible for detection by bioluminescence. This seemed to be the case since mice that harbored antiskin autoantibodies (14) had significantly increased light emissions from the ear (FIG. 4B, and supplemental Figure S2 at http://ajp.amjpathol.org).

Hitherto Unrecognized Affection of Small Intestine Revealed by Increased NF-κB Signals

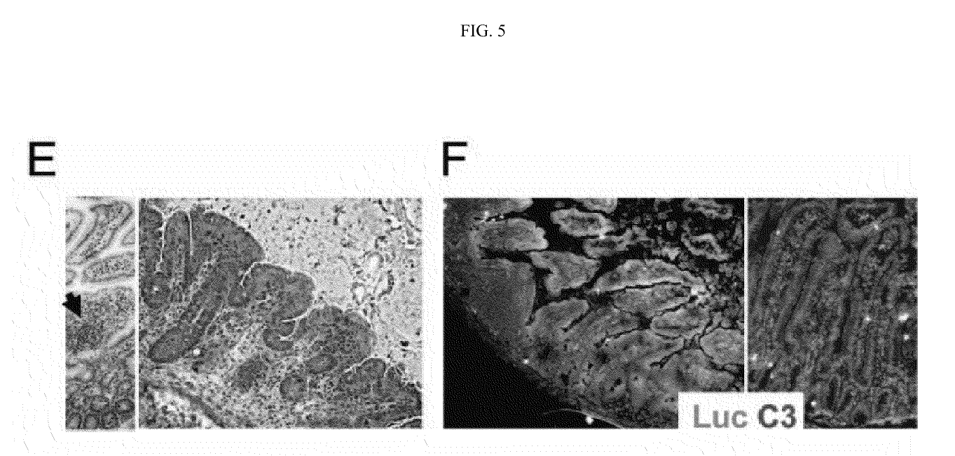

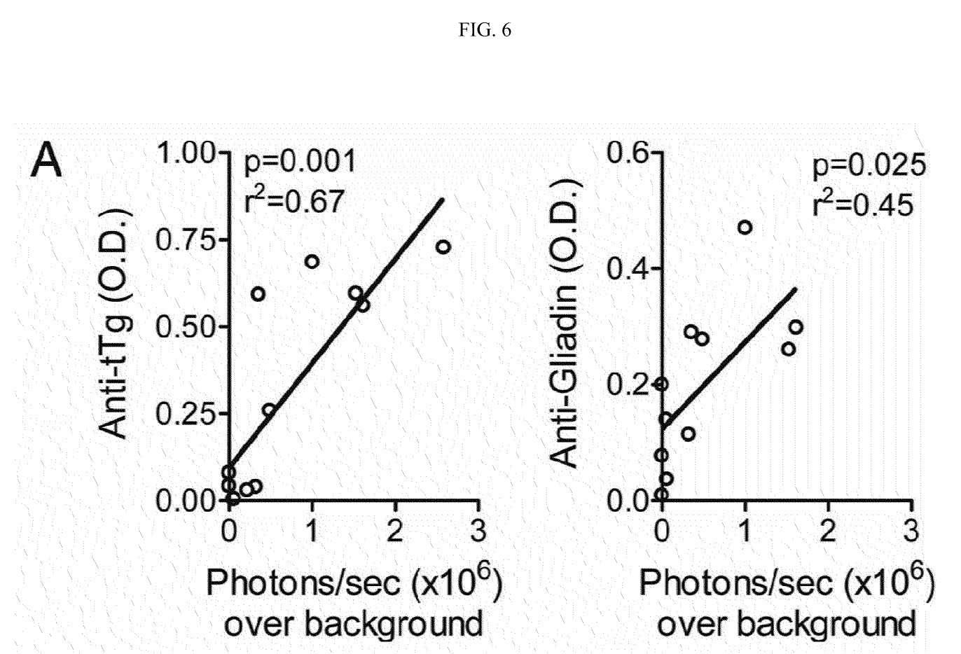

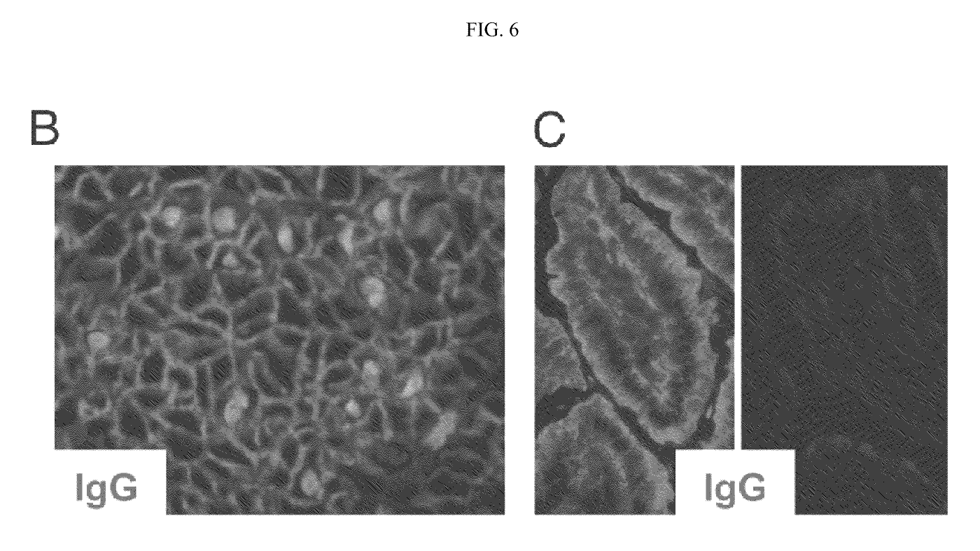

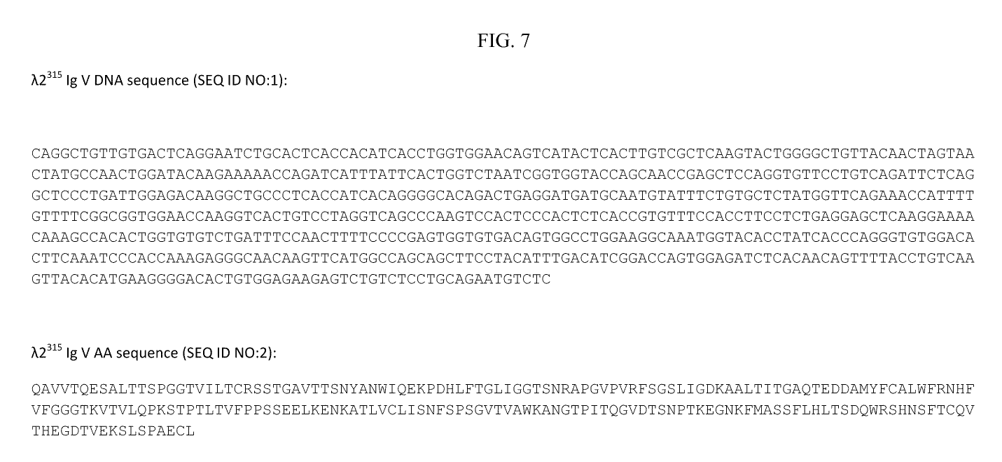

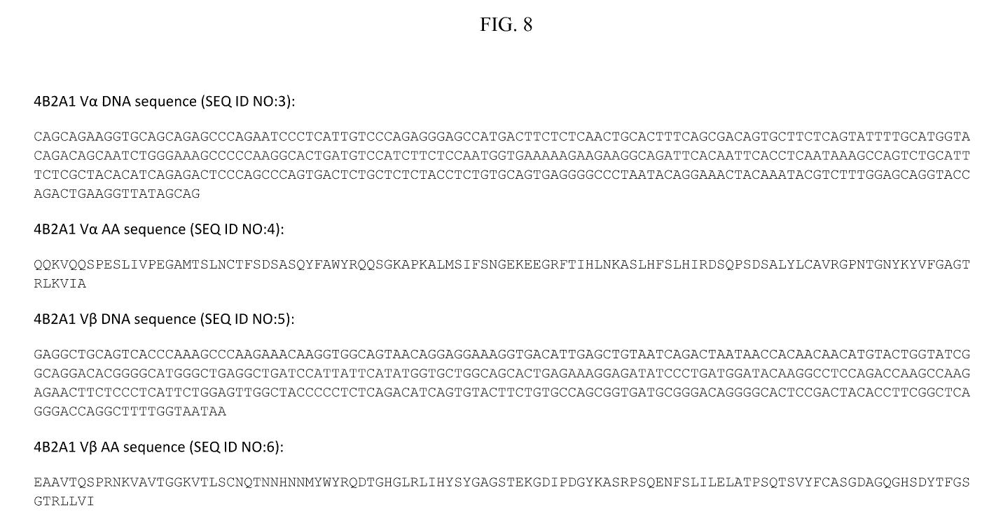

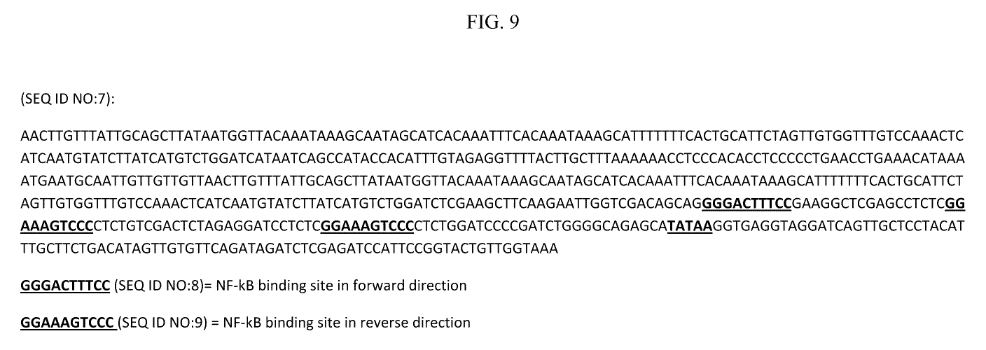

Small intestinal disease has not previously been described under conditions of chronic Id-driven T-B collaboration, and has only rarely been found in other mouse models of autoimmune diseases (29-31). It was therefore surprising to observe that 4 of the 13 triply transgenic mice had more than twice-increased photon emissions from the small bowel. The bioluminescence originated from both contiguous segments of the small intestine and from focal areas, the latter probably in part corresponding to increased activity in Peyers' patches (FIG. 2F). Interestingly, in three of these four mice, the light emissions from the small intestine were greater than the signals from the colons. Strikingly, these mice had serum IgG autoantibodies toward tissue transglutaminase (tTG2) and IgG anti-gluten antibodies (FIG. 6A) that correlated with signals from the small intestine. Moreover, two of the four had IgG antiendomycium autoantibodies (FIG. 6B). These parameters are all associated with celiac disease in humans. Further, two of these mice had low titers of serum IgG autoantibodies that stained murine small intestinal villi, including epithelial cells (FIG. 6C). Lastly, in addition to bioluminescence signals, the mice had inflammation in the small intestines, including cellular infiltrates, flattening of villi, C3 deposition and NF-κB/luciferase expression in a variety of cells (FIG. 5, E-F). Thus, the bioluminescence technology directed our attention to a previous overlooked area of pathology, the small intestine, where NF-κB activation correlated with autoantibodies, cellular infiltrates, and disease.

Discussion

We here use bioluminescence to detect and track systemic autoimmune disease in intact mice. The autoimmune disease in this model relies on the pathogenicity of so-called Id-driven T-B collaboration (13,14) where B cells present BCR V region antigenic determinants as Id peptides on MHC class II to Id-specific T cells. In such Id-driven T-B collaboration, autoreactive Id⁺ B cells receive help from Id-specific T cells, undergo the germinal center reaction, and secrete isotype-switched pathogenic autoantibodies (13,14). We have previously described that doubly transgenic offspring from (Id-specific TCR transgenic X Id⁺ L chain transgenic) mice develop systemic autoimmune disease with SLE-like features (14). Herein we introduced a NF-κB responsive luciferase reporter transgene (23) into the model to follow NF-κB activation in triply transgenic mice.

Importantly NF-κB signals were consistently detected several weeks before clinically manifest inflammatory bowel disease, skin disease, and arthritis. Moreover the NF-κB signals preceded the detection of autoantibodies. When disease developed, the clinical severity correlated with the bioluminescence signals. Finally, bioluminescence signals revealed sites of disease that went unnoticed by clinical inspection and autopsy alone. These results indicate that NF-κB mediated bioluminescence is a very sensitive and early indicator of inflammation and disease.

Bioluminescent imaging has a high sensitivity, since as few as 103 to 106 cells can be detected, depending on the anatomical site (32). The technique also has a high specificity, as background light emission is virtually negligible (33) and is low in cost and high-throughput (34). Although advances such as three-dimensional imaging techniques have been developed, and gene activation and expression can be monitored (as in the current paper), bioluminescent imaging is limited by a spatial resolution in the millimeter range.

Further, tissues vary in their absorption of light emissions, and pathological processes may influence tissue permeability to light, eg, increased blood content may cause increased light absorption by hemoglobin (35). Despite these limitations, the technology applied to disease models such as the one examined herein should be very useful for studies on early intervention, e.g., drug treatment, to prevent or treat autoimmune disease. Moreover, the technology might be extended to human disease in human 3 mouse xenograft models.

NF-κB activation can result from a plethora of cellular stimuli and signaling pathways (20,21,36). Thus NF-κB mediated light emissions could in the present experiments result from a number of independent pathways and settings, with contributions from (i) baseline, homeostatic NF-κB signals, (ii) Id-specific TCR signaling, (iii) autoantigen-specific BCR signaling, (iv) Id-driven T-B collaboration, and (v) autoantibody secretion, immune complex deposition, recruitment of cells to sites of inflammation, tissue destruction, erosions and exposure to intestinal microflora.

As concerns baseline homeostatic signaling (i) during the first 3 weeks of age, NF-κB signals from triply transgenic were confined to lymph nodes and the thymus and were equivalent to littermate controls. This background level of NF-κB could be caused by responses to homeostatic chemokines such as secondary lymphoid-tissue chemokine (SLC, CCL21), stromal cell-derived factor-1 (SDF-1, CXCL12) or to the TNF family member B-cell activating factor (BAFF) (37-39).

Id-specific TCR signaling (ii) probably contributed to the signal since we show that ligation of the Id-specific receptor induced distinct NF-κB signals (most likely via the classical pathway) in both in vitro assays and in adoptive transfer studies. Moreover, superficial lymph nodes (inguinal and cervical) gave strong signals in triple transgenic mice from 5 weeks of age. Interestingly, triple transgenic mice had a strong bioluminescent signal from the thymi even though thymi are quite small in these mice due to negative selection (10,14). Signaling of Id-specific thymocytes and induction of their apoptosis could be the cause of this signal.

Concerning autoantigen-specific BCR signaling and germinal center formation (iii), this would be expected to cause NF-κB activation (20,21,36). Consistent with this, we show that Id⁺ B cells express luciferase protein but we do not show whether the expression is due to ligation of their BCR by autoantigens or help from Id-specific T cells or both.

However, as antinuclear antibody levels correlated with NF-κB /light emissions and disease progression, the importance of BCR signaling for light emission is suggested. An interesting possibility is that anti-dsDNA Id⁺ B cells, which are frequent in triply transgenic mice (L. M., B. B., manuscript in preparation), can bind chromatin through both BCR and Toll-like receptor 9 (TLR9) (40,41). allowing for NF-κB activation via two separate stimulants of the classical pathway.

It would be expected from the two preceding paragraphs that Id-driven T-B collaboration (iv) significantly contributed to the NF-κB signal. Indeed, by development of a staining method for luciferase protein, we could show by immunohistochemistry that luciferase protein, and hence NF-κB activation, was found in Id⁺ B cells and Id-specific T cells. Moreover, Id-specific T cells positive for luciferase protein and Id⁺ B cells often interacted in clusters. Finally, interacting T and B cells in vitro produced a bioluminescence signal. These results demonstrate that Id-driven T-B collaboration directly contributed to light emissions.

As for secondary effects of autoantibody production like complement activation and inflammation (v), it is known that both immune complexes and complement factors activate NF-κB in phagocytes, allowing secretion of TNF, TNF-mediated NF-κB activation, and oxidative burst with release of oxygen radicals by phagocytes (20,21,42). Consistent with this, we found that colons with immune complex and complement deposition displayed intense NF-κB activation, and co-localized with phagocyte oxidative bursts visualized in vivo by use of a specific probe (L-012) (27). Moreover, mucosal erosions should expose cells in the underlying tissue to the luminal microflora. Indeed, such areas had intense microscopic expression of luciferase protein, probably in part due to activation of the classical NF-κB pathway by pattern recognition receptors (38).

We have previously described that Id-driven T-B collaboration results in a systemic disease with SLE-like features in addition to organ specific affections, eg, of the large bowel, skin, and joints (14). We here report a novel site of involvement, the small intestine, where bioluminescence signals were related to the presence of antibodies. With some exceptions such as graft versus host disease (30) and experiments with CD8 intraepithelial T cell epitopes (31), the search for murine models of human small intestinal disease has been difficult. However, multiple examples of inflammatory disease of the colon are well established (29). In the Id-driven T-B autoimmunity model herein, autoantibodies toward tTG2 and IgG anti-gliadin antibodies correlated with light emission from the small bowel. In humans, transglutimase is responsible for the deamidation of glutamine residues of wheat gliadin, a requirement for binding of gluten epitopes to HLA-DQ2 in celiac disease (43). tTG2 is increasingly expressed in inflammatory responses, and is up-regulated in cells undergoing apoptosis (44). Whether antibodies to gliadin and tTG2 are directly associated with induction of small intestinal disease, or are secondary phenomena in this model, remain to be elucidated.

1. Williams W M, Staines N A, Muller S, Isenberg D A: Human T cell responses to autoantibody variable region peptides. Lupus 1995, 4:464-471

2. Dayan M, Segal R, Sthoeger Z, Waisman A, Brosh N, Elkayam O, Eilat E, Fridkin M, Mozes E: Immune response of SLE patients to peptides based on the complementarity determining regions of a pathogenic anti-DNA monoclonal antibody. J Clin Immunol 2000, 20:187-194

3. van Schooten W C, Devereux D, Ho C H, Quan J, Aguilar B A, Rust C J: Joint-derived T cells in rheumatoid arthritis react with self-immunoglobulin heavy chains or immunoglobulin-binding proteins that copurify with immunoglobulin. Eur J Immunol 1994, 24:93-98

4. Holmoy T, Fredriksen A B, Thompson K M, Hestvik A L, Bogen B, Vartdal F: Cerebrospinal fluid T cell clones from patients with multiple sclerosis: recognition of idiotopes on monoclonal IgG secreted by autologous cerebrospinal fluid B cells. Eur J Immunol 2005, 35:1786-1794

5. Singh R R, Kumar V, Ebling F M, Southwood S, Sette A, Sercarz E E, Hahn B H: T cell determinants from autoantibodies to DNA can upregulate autoimmunity in murine systemic lupus erythematosus. J Exp Med 1995, 181:2017-2027

6. Singh R R, Hahn B H: Reciprocal T-B determinant spreading develops spontaneously in murine lupus: implications for pathogenesis. Immunol Rev 1998, 164:201-208

7. Zhang X, Smith D S, Guth A, Wysocki L J: A receptor presentation hypothesis for T cell help that recruits autoreactive B cells. J Immunol 2001, 166:1562-1571

8. Weiss S, Bogen B: B-lymphoma cells process and present their endogenous immunoglobulin to major histocompatibility complexrestricted T cells. Proc Natl Acad Sci USA 1989, 86:282-286

9. Weiss S, Bogen B: MHC class II-restricted presentation of intracellular antigen. Cell 1991, 64:767-776

10. Bogen B, Dembic Z, Weiss S: Clonal deletion of specific thymocytes by an immunoglobulin idiotype. EMBO J 1993, 12:357-363

11. Munthe L A, Kyte J A, Bogen B: Resting small B cells present endogenous immunoglobulin variable-region determinants to idiotope-specific CD4+ T cells in vivo. Eur J Immunol 1999, 29:4043-4052

12. Snyder C M, Zhang X, Wysocki L J: Negligible class II MHC presentation of B cell receptor-derived peptides by high density resting B cells. J Immunol 2002, 168:3865-3873

13. Munthe L A, Os A, Zangani M, Bogen B: MHC-restricted Ig V regiondriven T-B lymphocyte collaboration: B cell receptor ligation facilitates switch to IgG production. J Immunol 2004, 172:7476-7484

14. Munthe L A, Corthay A, Os A, Zangani M, Bogen B: Systemic autoimmune disease caused by autoreactive B cells that receive chronic help from Ig V region-specific T cells. J Immunol 2005, 175:2391-2400

15. Snyder C M, Aviszus K, Heiser R A, Tonkin D R, Guth A M, and Wysocki L J: Activation and tolerance in CD4+ T cells reactive to an immunoglobulin variable region. J Exp Med 2004, 200:1-11

16. Bogen B, Weiss S: Processing and presentation of idiotypes to MHCrestricted T cells. Int Rev Immunol 1993, 10:337-355

17. Bogen B, Jorgensen T, Hannestad K: T helper cell recognition of idiotopes on lambda 2 light chains of M315 and T952: evidence for dependence on somatic mutations in the third hypervariable region. Eur J Immunol 1985, 15:278-281

18. Bogen B, Malissen B, Haas W: Idiotope-specific T cell clones that recognize syngeneic immunoglobulin fragments in the context of class II molecules. Eur J Immunol 1986, 16:1373-1378

19. Eyerman M C, Zhang X, Wysocki L J: T cell recognition and tolerance of antibody diversity. J Immunol 1996, 157:1037-1046

20. Karin M, Lin A: NF-[kappa]B at the crossroads of life and death. Nat Immunol 2002, 3:221-227

21. Perkins N D: Integrating cell-signalling pathways with NF-kappaB and IKK function. Nat Rev Mol Cell Biol 2007, 8:49-62

22. Karin M, Ben-Neriah Y: Phosphorylation meets ubiquitination: the control of NF-kB activity. Ann Rev Immunol 2000, 18:621-663

23. Carlsen H, Moskaug J O, Fromm S H, Blomhoff R: In vivo imaging of NF-[kappa]B activity. J Immunol 2002, 168:1441-1446

24. Bogen B, Weiss S: A rearranged lambda 2 light gene chain retards but does not exclude kappa and lambda 1 expression. Eur J Immunol 1991, 21:2391-2395

25. Bogen B, Gleditsch L, Weiss S, Dembic Z: Weak positive selection of transgenic T cell receptor-bearing thymocytes: importance of major histocompatibility complex class II. T cell receptor and CD4 surface molecule densities Eur J Immunol 1992, 22:703-709

26. Bogen B, Lauritzsen G F, Weiss S: A stimulatory monoclonal antibody detecting T cell receptor diversity among idiotype-specific, major histocompatibility complex-restricted T cell clones. Eur J Immunol 1990, 20:2359-2362

27. Nishinaka Y, Aramaki Y, Yoshida H, Masuya H, Sugawara T, Ichimori Y: A new sensitive chemiluminescence probe. L-012, for measuring the production of superoxide anion by cells. Biochem Biophys Res Commun 1993, 193:554-559

28. Andrews B S, Eisenberg R A, Theofilopoulos A N, Izui S, Wilson C B, McConahey P J, Murphy E D, Roths J B, Dixon F J: Spontaneous murine lupus-like syndromes. Clinical and immunopathological manifestations in several strains. J Exp Med 1978, 148:1198-1215

29. Elson C O, Cong Y, McCracken V J, Dimmitt R A, Lorenz R G, Weaver C T: Experimental models of inflammatory bowel disease reveal innate, adaptive, and regulatory mechanisms of host dialogue with the microbiota. Immunol Rev 2005, 206:260-276

30. Cornelius E A: Protein-losing enteropathy in the graft-versus-host reaction. Transplantation 1970, 9:247-252

31. Vezys V, Lefrancois L: Inflammatory signals drive organ-specific autoimmunity to normally cross-tolerizing endogenous antigen. J Immunol 2002, 169:6677-6680

32. Edinger M, Sweeney T J, Tucker A A, Olomu A B, Negrin R S, Contag C H: Noninvasive assessment of tumor cell proliferation in animal models. Neoplasia 1999, 1:303-310

33. Hildebrandt I J, Gambhir S S: Molecular imaging applications for immunology. Clin Immunol 2004, 111:210-224

34. Kim S B, Ozawa T, Watanabe S, Umezawa Y: High-throughput sensing and noninvasive imaging of protein nuclear transport by using reconstitution of split Renilla luciferase. Proc Natl Acad Sci USA 2004, 101:11542-11547

35. Weissleder R: A clearer vision for in vivo imaging. Nature Biotechnol 2001, 19:316-317

36. Ruland J, Mak T W: Transducing signals from antigen receptors to nuclear factor kappaB. Immunol Rev 2003, 193:93-100

37. Schneider P: The role of APRIL and BAFF in lymphocyte activation. Curr Opin Immunol 2005, 17:282-289

38. Bonizzi G, Karin M: The two NF-kappaB activation pathways and their role in innate and adaptive immunity. Trends Immunol 2004, 25:280-288

39. Enzler T, Bonizzi G, Silverman G J, Otero D C, Widhopf G F, Anzelon-Mills A, Rickert R C, Karin M: Alternative and classical NF-kappa B signaling retain autoreactive B cells in the splenic marginal zone and result in lupus-like disease. Immunity 2006, 25:403-415

40. Leadbetter E A, Rifkin I R, Hohlbaum A M, Beaudette B C, Shlomchik M J, Marshak-Rothstein A: Chromatin-IgG complexes activate B cells by dual engagement of IgM and Toll-like receptors. Nature 2002,416:603-607

41. Christensen S R, Kashgarian M, Alexopoulou L, Flavell R A, Akira S, Shlomchik M J: Toll-like receptor 9 controls anti-DNA autoantibody production in murine lupus. J Exp Med 2005, 202:321-331

42. Bianchi G, Montecucco F, Bertolotto M, Dallegri F, Ottonello L: Immune complexes induce monocyte survival through defined intracellular pathways Ann NY Acad Sci 2007, 1095:209-219

43. Sollid L M: Coeliac disease: dissecting a complex inflammatory disorder. Nat Rev Immunol 2002, 2:647-655

44. Fesus L, Szondy Z: Transglutaminase 2 in the balance of cell death and survival. FEBS Lett 2005, 579:3297-3302

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of this invention are intended to be within the scope of the following claims.

What is claimed is:

1. A transgenic mouse comprising an Id⁺ L chain transgene, an Id-specific TCR transgene, and a reporter transgene comprising NF-κB responsive promoter elements operably linked to a reporter gene.

2. The transgenic mouse of claim 1, wherein said reporter gene encodes a bioluminescent or fluorescent protein.

3. The transgenic mouse of claim 1, wherein said Id⁺ L chain transgene encodes the λ2³¹⁵ Ig L-chain.

4. The transgenic mouse of claim 1, wherein said Id-specific TCR transgene encodes the αβ TCR specific for an Id(λ2³¹⁵)-peptide.

5. The transgenic mouse of claim 1, wherein said reporter gene is a luciferase gene.

6. The transgenic mouse of claim 1, wherein said mouse is homozygous for said Id⁺ L chain transgene, said Id-specific TCR transgene, and said reporter transgene.

7. A method of producing transgenic mice comprising an Id⁺ L chain transgene, an Id-specific TCR transgene, and a reporter transgene comprising an NF-κB responsive promoter elements operably linked to a reporter gene comprising crossing a mouse line comprising an Id⁺ L chain transgene and an Id-specific TCR transgene with a mouse line comprising a reporter transgene comprising an NF-κB responsive promoter elements operably linked to a reporter gene.

8. A transgenic mouse produced by the method of claim 7.

9. A method of testing a compound comprising:
contacting a transgenic mouse comprising an Id⁺ L chain transgene, an Id-specific TCR transgene, and a reporter transgene comprising NF-κB responsive promoter elements operably linked to a reporter gene with a test compound, and assaying expression of said reporter gene.

10. The method of claim 9, wherein said test compound is a drug.

11. The method of claim 9, further comprising contacting said transgenic mouse with a plurality of test compounds.

12. The method of claim 11, wherein said plurality of test compounds are a combinatorial library of compounds.

13. The method of claim 9, wherein said test compound is a small organic compound.

14. The method of claim 9, wherein aid test compound is a peptide or protein.

15. The method of claim 9, further comprising using said assay results to evaluate said compound for treatment of an autoimmune disease.

16. The method of claim 15, wherein said autoimmune disease is a skin disease, small intestine disease, kidney disease, large intestine disease, or arthritis.

17. The method of claim 9, further comprising the step of identifying a candidate drug and testing said candidate drug in a human.

18. The method of claim 17, further comprising providing said candidate drug to human patients.

19. A method of assessing progression of an autoimmune disease comprising:
providing a transgenic mouse comprising an Id⁺ L chain transgene, an Id-specific TCR transgene, and a reporter transgene comprising NF-κB responsive promoter elements operably linked to a reporter gene with an experimental treatment, and
assaying expression of said reporter gene.

20. The method of claim 19, wherein said transgenic mouse is exposed to an experimental treatment.
